(19) United States
(12) Patent Application Publication
(10) Pub. No.: US 2021/0186461 A1
(43) Pub. Date: Jun. 24, 2021

(54) CONVEX-TYPE ULTRASOUND PROBE

(71) Applicant: HITACHI, LTD., Chiyoda-ku, Tokyo (JP)

(72) Inventors: Hidetsugu KATSURA, Chiyoda-ku, Tokyo (JP); Tomohiro SHIRAISHI, Chiyoda-ku, Tokyo (JP); Takayuki IWASHITA, Chiyoda-ku, Tokyo (JP)

(73) Assignee: HITACHI, LTD., Chiyoda-ku, Tokyo (JP)

(21) Appl. No.: 16/622,135

(22) PCT Filed: May 18, 2018
(86) PCT No.: PCT/JP2018/019383

§ 371 Date: Dec. 12, 2019

(30) Foreign Application Priority Data
Jun. 13, 2017 (JP) 2017-115993

(51) Int. Cl. A61B 8/00; B06B 1/06; H01L 41/04; H01L 41/047; G01S 15/89

(52) U.S. Cl. CPC A61B 8/4455; B06B 1/0622; H01L 41/042; B06B 2201/76; A61B 8/4494; G01S 15/892; H01L 41/0475

(57) ABSTRACT

A backing 20 includes a lead array 24a that electrically connects each vibration element 12 and a plurality of ICs 32. Each bump 42 provided at a lower end portion of each lead 24 is connected to a conductor pad on an upper side surface of a relay substrate 26, and a ball-shaped terminal 44 of each IC 32 is connected to a lower surface of the relay substrate 26. The lower end portions of the leads 24 are grouped into a plurality of dense groups 46 corresponding to each IC 32 in a longitudinal direction (X-axis direction) by wiring patterns of the leads.

### TECHNICAL FIELD

The present invention relates to a convex ultrasonic probe. Particularly, the invention relates to a convex ultrasonic probe that includes a backing including a plurality of leads that electrically connect a plurality of vibration elements that are arranged two-dimensionally and a plurality of electronic devices.

### BACKGROUND ART

An ultrasonic diagnostic device is used in a medical field. The ultrasonic diagnostic device is a device that transmits and receives an ultrasonic wave to and from a test object, and forms an ultrasonic image based on a reception signal obtained thereby. The ultrasonic wave is transmitted and received by an ultrasonic probe connected to a device main body.

Various types of ultrasonic probes are known, including a convex ultrasonic probe. In the convex ultrasonic probe, a plurality of vibration elements are arranged in an arc shape in one direction (usually longitudinal direction) of two-dimensional arrangement directions of a vibration element array, and an surface of the vibration element array has a curved shape (U shape). By using the convex ultrasonic probe, it is possible to observe with a wide angle at a deep portion of an ultrasonic irradiation region while keeping a certain field of view in a shallow portion of the ultrasonic irradiation region.

An ultrasonic probe including the convex ultrasonic probe may have a configuration including a vibration element array that includes a plurality of vibration elements and transmits and receives an ultrasonic wave, a backing that is provided on a lower side (side opposite to a transmission and reception surface) of the vibration element array and prevents excessive vibration of the vibration element array, and an electronic device further provided on a lower side of the backing. Examples of the electronic device include an IC that exhibits a channel reduction function for reducing the number of wirings contained in a cable that connects an ultrasonic probe and a device main body.

In the related art, in an ultrasonic probe having the above-described configuration, a backing including a plurality of leads (conductive wires) that electrically connect a plurality of vibration elements and an electronic device is proposed (for example, PTL 1). As disclosed in PTL 1, in a two-dimensional ultrasonic probe in which a vibration element array is two-dimensionally arranged, the plurality of leads of the backing are also two-dimensionally arranged. Further, in the related art, also in a convex two-dimensional ultrasonic probe, a backing including a plurality of leads that electrically connect a plurality of vibration elements and an electronic device is used (for example, PTL 2 and PTL 3).

### PRIOR ART LITERATURE

### Patent Literature

PTL 1: JP-A-2015-228932

PTL 2: JP-T-2008-526343

PTL 3: JP-A-2002-28159

### SUMMARY OF INVENTION

### Technical Problem

In an ultrasonic probe including a plurality of vibration elements that are arranged two-dimensionally, a backing including a plurality of leads and an electronic device, a relay substrate may be further provided between the backing including the plurality of leads and the electronic device. For example, after the electronic device is mounted on the relay substrate, the relay substrate and the plurality of leads of the backing are electrically connected to each other. Accordingly, the plurality of leads of the backing and the electronic device are electrically connected to each other via the relay substrate.

Here, a connection position of each of the plurality of leads of the backing with respect to the relay substrate and a terminal position of each terminal of the electronic device with respect to the relay substrate do not correspond to each other, which may cause a problem that a wiring pattern of the relay substrate may be complicated. For example, considering a case where the plurality of leads of the backing are connected to an upper side surface of the relay substrate and the electronic device is mounted on a lower side surface of the relay substrate, when the connection position of each of the plurality of leads on the upper side surface of the relay substrate and the terminal position of each terminal of the electronic device on the lower side surface of the relay substrate do not correspond to each other, the wiring pattern of the relay substrate is long and complicated so as to correct a deviation between the connection position and the terminal position. Particularly, the problem is significant when a plurality of electronic devices are provided.

An object of the invention is to simplify a wiring pattern of a relay substrate in a convex ultrasonic probe that includes a plurality of vibration elements that are arranged two-dimensionally, a backing including a plurality of leads, a plurality of electronic devices, and a relay substrate provided between the backing and the plurality of electronic devices.

### Solution to Problem

The invention provides a convex ultrasonic probe including a vibration element array that includes a plurality of vibration elements that are arranged two-dimensionally in a curved direction corresponding to a longitudinal direction and a short direction perpendicular to the longitudinal direction; a backing that is provided on a lower side of the vibration element array and includes a lead array including a plurality of leads electrically connected to the plurality of vibration elements, wherein an upper side surface of the backing is a curved surface defined by the curved direction and the short direction; a plurality of electronic devices that are provided on a lower side of the backing; and a relay substrate that is a substrate extending in the longitudinal direction and the short direction and electrically connects the lead array and the plurality of electronic devices, in which lower end portions of the plurality of leads are grouped into a plurality of dense groups according to an arrangement of the plurality of electronic devices at least in the longitudinal direction.

According to the above-described configuration, the lower end portions of the plurality of leads of the backing are grouped into the plurality of dense groups according to the arrangement of the electronic devices at least in the longitudinal direction. That is, the lower end portion of each lead is gathered at a position corresponding to each electronic device. In the related art, when a deviation between a contact position of each lead of the backing and the relay substrate and a position (terminal) of the electronic device is corrected by a wiring pattern of the relay substrate, at least a length of the wiring pattern of a relay substrate 26 in the longitudinal direction can be reduced by grouping. That is, the wiring pattern of the relay substrate 26 is simplified. As described above, in the above-described configuration, each lead of the backing at least supplementarily corrects the deviation between the contact position of each lead of the backing and the relay substrate, and the position of the electronic device.

Preferably, an inter-group gap exists between two adjacent dense groups in the longitudinal direction, an inter-device gap exists between two adjacent electronic devices in the longitudinal direction, and an arrangement of a plurality of inter-group gaps existing on an upper side of the relay substrate corresponds to an arrangement of a plurality of inter-device gaps on a lower side of the relay substrate.

Preferably, the lead array includes a plurality of lead rows arranged in the short direction, each of the lead rows includes a plurality of leads arranged in the longitudinal direction, and a pitch between the plurality of lead rows in the short direction is constant.

In the convex ultrasonic probe, a length in the short direction is usually considerably shorter than a length in the longitudinal direction. Therefore, since the wiring pattern of the relay substrate in the longitudinal direction is likely to be long, that is, is likely to be complicated, it can be said that a demand for simplification in the longitudinal direction is particularly strong. On the other hand, difficulty of manufacturing the backing may be increased by grouping the plurality of leads not only in the longitudinal direction but also in the short direction. Therefore, by grouping the plurality of leads in the longitudinal direction and making the pitch between the leads constant in the short direction (not grouping) , simplification of the relay substrate in the longitudinal direction is achieved, and the difficulty of manufacturing the backing can be kept low. For example, by making the pitch of each lead in the short direction constant, it is possible to adopt a method of laminating a lead sheet in which the plurality of leads arranged in the longitudinal direction are embedded in a sheet-shaped backing base portion at the time of manufacturing the backing, and according to the method, the backing can be easily formed.

Preferably, each of the lead rows includes a plurality of sections arranged in an upper-lower direction with different wiring patterns, and a plurality of dense groups are formed by the wiring pattern in any one of the plurality of sections.

Preferably, the plurality of sections includes an upper end section that is an upper end portion of a lead row and has a radial pattern corresponding to the vibration element array; an intermediate section that is an intermediate portion of the lead row in the upper-lower direction and has a main wiring pattern which is a parallel wiring pattern; a lower end section that is a lower end portion of the lead row and has a grouping pattern including the plurality of dense groups which is a parallel wiring pattern; an upper transition section that is a section between the upper end section and the intermediate section and has an upper transition pattern that connects the radial pattern and the main wiring pattern; and a lower transition section that is a section between the intermediate section and the lower end section and has a lower transition pattern that connects the main wiring pattern and the grouping pattern.

Preferably, the radial pattern includes a plurality of lead upper end portions perpendicular to the curved surface. Preferably, the grouping pattern includes a plurality of lead lower end portions perpendicular to a horizontal plane defined by the longitudinal direction and the short direction.

Preferably, the plurality of lead lower end portions are grouped into a plurality of dense groups according to the arrangement of the plurality of electronic devices also in the short direction.

Preferably, the vibration element array is formed on an intermediate portion excluding both end portions of an upper side surface of the backing, and an electrode sheet that is laminated on an upper side of the vibration element array and is electrically connected to the leads at both ends of the upper side surface of the backing is provided. According to the configuration, the electrode sheet laminated on the upper side of the vibration element array can be electrically connected to the relay substrate via the lead. Accordingly, for example, the electrode sheet can be suitably connected to a ground potential.

### Advantageous Effect

According to the invention, in the convex ultrasonic probe that includes a plurality of vibration elements that are arranged two-dimensionally, a backing including a plurality of leads, a plurality of electronic devices, and a relay substrate provided between the backing and the plurality of electronic devices, the wiring pattern of the relay substrate can be simplified.

### BRIEF DESCRIPTION OF DRAWINGS

FIG. 1 is a partial cross-sectional perspective view of a vibrator unit according to an embodiment.

FIG. 2 is a front cross-sectional view of a backing, a relay substrate, and ICs.

FIG. 3 is a side cross-sectional view of the backing, the relay substrate, and the ICs.

FIG. 4 is a horizontal cross-sectional view of the backing.

FIG. 5 is an enlarged view of an end portion in an X-axis direction which is an upper end portion of the backing.

### DESCRIPTION OF EMBODIMENTS

Hereinafter, an ultrasonic probe according to an embodiment will be described. The ultrasonic probe according to the present embodiment is connected to an ultrasonic diagnostic device and transmits and receives an ultrasonic wave to and from a test object. The ultrasonic probe according to the present embodiment is a convex two-dimensional ultrasonic probe.

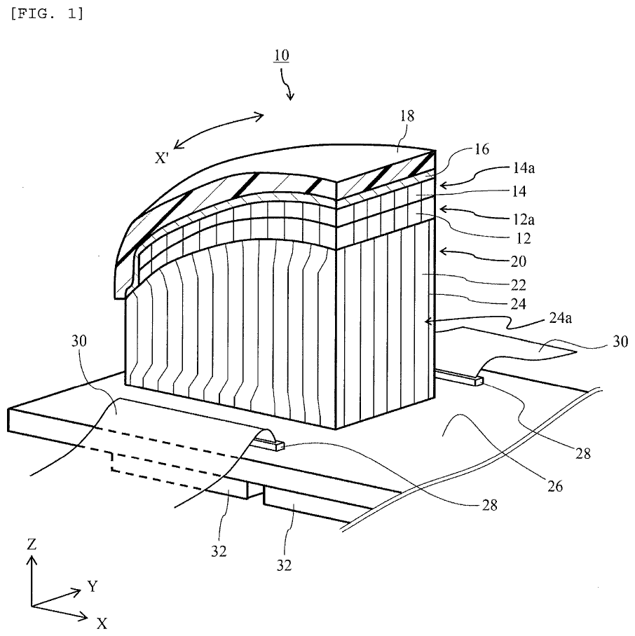

FIG. 1 is a partial cross-sectional perspective view of a vibrator unit 10 incorporated in the ultrasonic probe according to the present embodiment.

As shown in FIG. 1, the vibrator unit 10 is formed by laminating members. In FIGS. 1 to 5, a lamination direction of the members in the vibrator unit 10 is defined as a Z-axis direction and directions orthogonal to the Z-axis are an X-axis direction and a Y-axis direction. In FIG. 1, an ultrasonic wave is transmitted toward a Z-axis positive direction side. That is, the Z-axis positive direction side is a transmission and reception surface side of the ultrasonic wave (test object side). In this specification, the Z-axis positive direction side is referred to as an “upper side” and a Z axis lower direction side is referred to as a “lower side”. A plane defined by the X axis and the Y axis is referred to as a “horizontal plane”. As a matter of course, since a posture of the ultrasonic probe changes, the terms “upper side”, “lower side”, and “horizontal plane” in this specification indicate relative directions or planes.

A vibration element array 12a is configured by two-dimensionally arranging a plurality of vibration elements 12. As described above, since the ultrasonic probe according to the present embodiment is a convex two-dimensional ultrasonic probe, the plurality of vibration elements 12 are two-dimensionally arranged in a curved direction (direction indicated by X′ in FIG. 1) corresponding to the X-axis direction and the Y-axis direction. In the present embodiment, the vibration element array 12a according to the present embodiment has a rectangular shape in a plan view, that is, the X-axis direction is a longitudinal direction, and the Y-axis direction is a short direction. In the present embodiment, one hundred and tens of vibration elements 12 are arranged in the curved direction, and several tens of vibration elements 12 are arranged in the short direction.

Each vibration element 12 is formed of such as a ceramic such as PZT (zircon and lead titanate) or a single crystal such as PMT-PT (lead magnesium niobate and lead titanate solid solution). A signal electrode (hereinafter referred to as a “lower electrode”) is provided on a lower side surface of each vibration element 12. A signal electrode (hereinafter, referred to as an “upper electrode”) is also provided on an upper side surface of each vibration element 12. In the present embodiment, the upper electrode of each vibration element 12 is connected to a ground potential, and a drive signal is applied to the lower electrode of each vibration element 12. Accordingly, each of the vibration elements 12 vibrates. When the drive signal is supplied to each vibration element 12, each vibration element 12 vibrates and an ultrasonic beam is transmitted. Each vibration element 12 receives a reflected echo that is reflected from the test object, and outputs a reception signal based on the received reflected echo.

An acoustic matching layer 14a laminated on the upper side of the vibration element array 12a is provided to prevent reflection of an ultrasonic wave on a surface of the test object by matching acoustic impedances between the vibration elements 12 and the test object. The acoustic matching layer 14a includes a plurality of acoustic matching elements 14 corresponding to the vibration elements 12. The acoustic matching layer 14a is formed of, for example, a resin, a carbon atom, and a carbon. The acoustic matching layer 14a also has a curved shape in accordance with the curved shape of the vibration element array 12a. Although only one acoustic matching layer 14a is shown in FIG. 1, the acoustic matching layer 14a may be configured with a plurality of layers.

An electrode sheet 16 is laminated on an upper side of the acoustic matching layer 14a. The electrode sheet 16 is formed of, for example, a metal film such as a copper foil. The electrode sheet 16 is connected to a ground potential by a method to be described later, and is in contact with an upper side surface of the acoustic matching layer 14a (each acoustic matching element 14). As described above, since the acoustic matching layer 14a is a conductor, the upper electrode of each vibration element 12 is connected to the ground potential by laminating the electrode sheet 16 on an upper side of the acoustic matching layer 14a.

A protective layer 18 is laminated on the upper side of the electrode sheet 16. The protective layer 18 protects layers below the acoustic matching layer 14a. The protective layer 18 is formed of, for example, silicone rubber. The protective layer 18 also has a curved shape in accordance with the vibration element array 12a and the acoustic matching layer 14a which have the curved shape. An upper side surface of the protective layer 18 is a surface to be in contact with the test object, that is, a transmission and reception surface.

A backing 20 is provided on a lower side of the vibration element array 12a. The backing 20 includes a backing base portion 22 that prevents unnecessary vibration of the vibration element array 12a, and a lead array 24a that is formed of a plurality of leads 24 that electrically connect the lower electrodes of the vibrating elements 12 and a relay substrate 26 to be described later. In accordance with the vibration element array 12a having a curved shape, an upper side surface of the backing 20 is a curved surface defined by the curved direction (direction indicated by an arrow X′) and the short direction (Y-axis direction).

The backing base portion 22 is formed by mixing a damping material filler with a resin, for example, epoxy, urethane, or acrylic. The damping material filler is formed of a ceramic or a metal, for example, tungsten.

The plurality of leads 24 are two-dimensionally arranged in the X-axis direction and the Y-axis direction in accordance with the two-dimensional arrangement of the vibration elements 12. Each of the plurality of leads 24 is electrically connected to the vibration element 12 on an upper end portion thereof, and is electrically connected to the relay substrate on a lower end portion thereof. Each lead 24 may be formed of a metal such as copper or phosphor bronze, but from the viewpoint of further reducing crosstalk between the leads 24, a material between the leads 24 is preferably formed of a material having a low dielectric constant, for example, a polymer material such as epoxy or polyimide.

The details of the backing 20, particularly, a wiring pattern of the leads 24 will be described later.

In FIG. 1, an XZ cross-section and a YZ cross-section of a laminated body including the backing 20, the vibration element array 12a, the acoustic matching layer 14a, the electrode sheet 16, and the protective layer 18 are shown. In FIG. 1, hatching of the backing 20, the vibration element array 12a, and the acoustic matching layer 14a is omitted (the same applies to the subsequent drawings).

The relay substrate 26 extending in the horizontal plane is provided on a lower side of the backing 20. The relay substrate 26 is a multilayer build-up substrate, and is a hard substrate formed of, for example, glass epoxy having a low dielectric constant. Alternatively, the relay substrate 26 may be a rigid flexible substrate in which a flexible cable is sandwiched by a rigid substrate.

A plurality of conductor pads are provided on an upper side surface of the relay substrate 26, and the conductor pads and the leads 24 are electrically connected to each other. Connectors 28 are provided on side end portions of the upper side surface of the relay substrate 26. A flexible cable 30 is connected to the connectors 28. The flexible cable 30 is connected to a wire in a cable connected to an ultrasonic diagnostic device via a connector (not shown). That is, the relay substrate 26 (that is, an IC 32 to be described later) is electrically connected to an ultrasonic diagnostic device main body by the flexible cable 30.

A plurality of ICs 32 serving as a plurality of electronic devices are mounted on a lower side surface of the relay substrate 26. Accordingly, the relay substrate 26 serves as a substrate that relays an electrical connection between the vibration elements 12 or the lead array 24a and the plurality of ICs 32. The IC 32 functions as a transmission sub-beamformer and a reception sub-beamformer. As the transmission sub-beam former, the IC 32 transmits a drive signal to the plurality of vibration elements 12 based on a transmission signal from the ultrasonic diagnostic device main body. As the reception sub-beamformer, the IC 32 performs phasing addition processing on reception signals from the plurality of vibration elements 12, generates a processed reception signal, and transmits the processed reception signal to the ultrasonic diagnostic device main body. As described above, the IC 32 exhibits a function (channel reduction function) for reducing the number of signal lines between the vibration element array 12a and the ultrasonic diagnostic device main body.

An outline of the vibrator unit 10 according to the present embodiment is as described above. Hereinafter, the backing 20, the relay substrate 26, and the IC 32 will be described in detail.

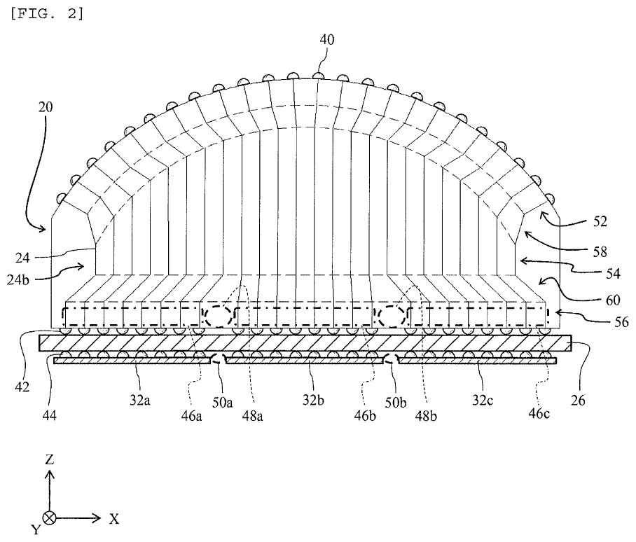

FIG. 2 shows a front cross-sectional view (XZ cross-sectional view) of the backing 20, the relay substrate 26, and the ICs 32. As shown in FIG. 1, the plurality of leads 24 are arranged two-dimensionally, and in FIG. 2, one lead row 24b including a plurality of leads 24 arranged in the longitudinal direction (X-axis direction) is shown. By arranging the lead row 24b as shown in FIG. 2 in the short direction (Y-axis direction) , the lead array 24a in which the plurality of leads 24 are two-dimensionally arranged is formed.

On an upper curved surface of the backing 20, a plurality of bumps 40 electrically connected to the leads 24 are formed. The bump 40 is a protruding portion formed of a metal and protruding from a surface of the upper curved surface, and is provided to further ensure electrical contact between each lead 24 and the lower electrode of each vibration element 12. Similarly, on a lower horizontal surface of the backing 20, a plurality of bumps 42 electrically connected to the leads 24 are formed. The bump 42 is also a protruding portion formed of a metal and protruding from a surface of the lower horizontal surface, and is provided to further ensure electrical contact between each lead 24 and the conductor pad provided on the upper side surface of the relay substrate 26.

In the present embodiment, the IC 32 is a surface mount type package, and the IC 32 has a plurality of ball-shaped terminals 44 on an upper side surface thereof. The plurality of ball-shaped terminals 44 are connected to the conductor pads provided on the lower side surface of the relay substrate 26 by a method such as soldering, so that the IC 32 is mounted on the relay substrate 26. In the present embodiment, six ICs 32 are provided. That is, as shown in FIG. 2, an IC row including three ICs 32 arranged in the longitudinal direction is formed, and two such IC rows are aligned in the short direction. As a matter of course, the number of ICs 32 may be changed as appropriate according to the number of the vibration elements 12 or the characteristics of each IC 32.

Due to a difference between a pitch between the vibration elements 12 in the longitudinal direction and a pitch between the ball-shaped terminals 44 of the IC 32, or the like, when the leads 24 of the backing 20 are linearly extended in an upper-lower direction (Z-axis direction), the contact position of each lead 24 and the relay substrate 26 (it can also be said a lower end position of each lead 24 and a position of each bump 42) and a position of each ball-shaped terminal 44 do not correspond to each other, and a deviation between both positions needs to be corrected in the wiring pattern of the relay substrate 26, and as a result, the wiring pattern of the relay substrate 26 is complicated. Therefore, in the present embodiment, a lower end position of each lead 24 in at least the longitudinal direction is set to a position corresponding to the position of each IC 32 (each ball-shaped terminal 44) by the wiring pattern of the lead array 24a. That is, in the present embodiment, each lead 24 of the backing 20 supplementarily corrects the deviation between the lower end position of each lead 24 and the position of each ball-shaped terminal 44. Details will be described below.

In the present embodiment, in the backing 20, the lower end portions of the plurality of leads 24 contained in the lead rows 24b are grouped into a plurality of dense groups 46 according to the arrangement of the ICs 32 in the longitudinal direction. In FIG. 2, the lower end portions of the plurality of leads 24 are grouped into three dense groups 46, a dense group 46a, a dense group 46b, and a dense group 46c. Each dense group 46 corresponds to each IC 32. That is, the dense group 46a corresponds to an IC 32a, the dense group 46b corresponds to an IC 32b, and the dense group 46c corresponds to an IC 32c.

Grouping means that the lower end portions of the plurality of leads 24 belonging to the same dense group 46 are disposed close to each other, and are disposed to be isolated from the lower end portions of the leads 24 belonging to another dense group. By grouping the lower end portions of the plurality of leads 24, inter-group gaps 48 are generated between the dense groups 46 at the lower end portions of the plurality of leads 24. Specifically, as shown in FIG. 2, an inter-group gap 48a is generated between the dense group 46a and the dense group 46b, and an inter-group gap 48b is generated between the dense group 46b and the dense group 46c.

Although the inter-group gaps 48 are necessarily arranged in the longitudinal direction, an arrangement of the inter-group gaps 48 corresponds to an arrangement of inter-device gaps 50 in the longitudinal direction. The inter-device gaps 50 similarly exist between the ICs 32 arranged in the longitudinal direction. Specifically, the inter-group gap 48a corresponds to an inter-device gap 50a, and the inter-group gap 48b corresponds to an inter-device gap 50b. The inter-group gap 48 may not necessarily be located directly above the corresponding inter-device gap 50.

Grouping of the lower end portions of the plurality of leads 24 is realized by the wiring pattern of the leads 24. Specifically, as shown in FIG. 2, the lead row 24b includes a plurality of sections (regions) arranged in the upper-lower direction. That is, the lead row 24b includes an upper end section 52 which is an upper end portion of the lead row 24b, an intermediate section 54 which is an intermediate portion of the lead row 24b in the upper-lower direction, a lower end section 56 which is a lower end portion of the lead row 24b, an upper transition section 58 that is provided between the upper end section 52 and the intermediate section 54 and a lower transition section 60 that is provided between the intermediate section 54 and the lower end section 56. The lower end portions of the leads 24 are grouped into a plurality of dense groups 46 by the wiring pattern of any one of the plurality of sections of the lead row 24b.

As described above, the upper side surface of the backing 20 is a curved surface, and an upper end of the backing 20 is curved in an arc shape in the XZ cross-section as shown in FIG. 2. The upper end section 52 has a radial pattern in which a plurality of lead portions (lead upper end portions) are arranged radially according to the curved surface of the backing 20. Specifically, each lead portion contained in the upper end section 52 extends in a direction perpendicular to the curved surface of the backing 20. Since the pitch between the vibration elements 12 in the curved direction is constant, the pitch between the lead upper end portions contained in the radial pattern in the upper end section 52 is also constant.

The intermediate section 54 has a parallel wiring pattern in which a plurality of lead portions are arranged in parallel in the upper-lower direction (Z-axis direction). The intermediate section 54 is a section having a longer wiring length than other sections, that is, the parallel wiring pattern contained in the intermediate section 54 is the main wiring pattern. In the intermediate section 54, the pitch between the lead portions contained in the main wiring pattern is preferably constant. Accordingly, crosstalk generated between the lead portions can be reduced as an overall intermediate section 54.

The lower end section 56 has a parallel wiring pattern in which the plurality of lead portions (lead lower end portions) are arranged perpendicular to the horizontal plane which is the lower side surface of the backing 20, that is, parallel in the upper-lower direction (Z-axis direction). As shown in FIG. 2, the lower end section 56 includes the plurality of dense groups 46. That is, the lower end section 56 has a grouping pattern including the plurality of dense groups 46 formed by the parallel wiring pattern. The pitch between the lead portions in the lower end section 56 and the pitch between the lead portions in the intermediate section 54 may be different from each other.

The upper transition section 58 has an upper transition pattern formed by the plurality of lead portions that respectively connect the lower end of each lead portion contained in the radial pattern of the upper end section 52 and the upper end of each lead portion contained in the main wiring pattern of the intermediate section 54.

The lower transition section 60 has a lower transition pattern formed by the plurality of lead portions that respectively connect the lower end of each lead portion contained in the main wiring pattern of the intermediate section 54 and the upper end of each lead portion contained in the grouping pattern of the lower end section 56.

In the present embodiment, in the intermediate section 54, an interval between the lead portions contained in the main wiring pattern is constant, whereas in the lower end section 56, the plurality of dense groups 46 are formed so as to correspond to the ICs 36. That is, in the present embodiment, grouping of the lower end portions of the leads into the dense groups 46 is realized by the lower transition pattern in the lower transition section 60 that connects the intermediate section 54 and the lower end section 56.

Although only one lead row 24b is shown in FIG. 2, similarly for others lead rows 24b arranged in the short direction, the lower end portions of the plurality of leads 24 are grouped so as to correspond to the respective ICs 32.

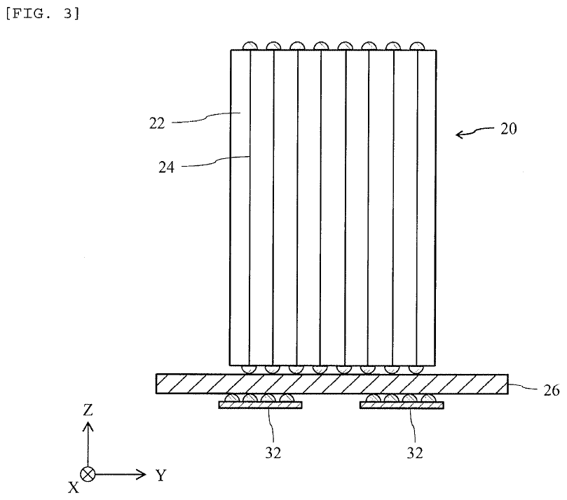

FIG. 3 shows a side cross-sectional view (YZ cross-sectional view) of the backing 20, the relay substrate 26, and the ICs 32. As shown in FIG. 3, in the present embodiment, the pitch between the leads 24 in the short direction is constant, that is, the leads 24 are not grouped corresponding to the ICs 32 in the short direction, but for the short direction, the lower end portions of the leads 24 may also be grouped corresponding to the arrangement of the ICs 32 in the short direction. In the present embodiment, the deviation between the lower end position of each lead 24 in the short direction and the position of each ball-shaped terminal 44 is corrected by the wiring pattern of the relay substrate 26.

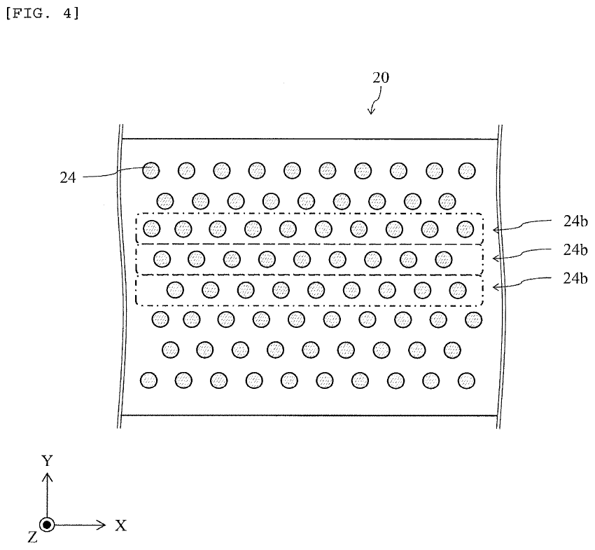

FIG. 4 shows a horizontal cross-sectional view of the intermediate section 54 of the backing 20. As described above, in the lead array 24a, the lead rows 24b are arranged in the short direction, and in at least the intermediate section 54, the positions in the longitudinal direction (X-axis direction) of the leads 24 contained in an adjacent lead rows 24b are different from each other. That is, the plurality of leads 24 in at least the intermediate section 54 are in a staggered arrangement. Accordingly, a distance between the adjacent leads 24 can be increased, and the crosstalk between the leads 24 can be reduced.

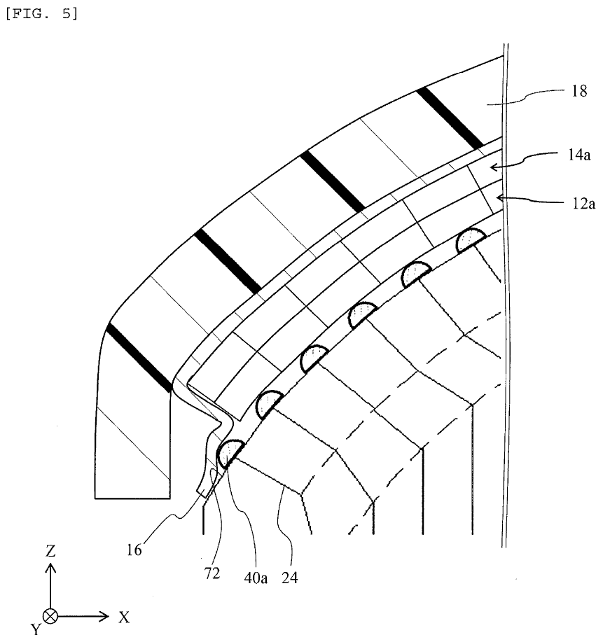

FIG. 5 shows an enlarged view of an end portion in the longitudinal direction which is an upper end portion of the backing 20. The vibration element array 12a and the acoustic matching layer 14a are laminated on an upper side of an intermediate portion excluding both end portions in the longitudinal direction on the upper side surface of the backing 20. That is, as shown in FIG. 5, the upper side surface of the backing 20 has, on an end portion thereof in the longitudinal direction, an exposed portion 72 on which the vibration element array 12a and the acoustic matching layer 14a are not laminated. Also in the exposed portion 72, a bump 40a connected to the lead 24 is formed. The bump 40a and the lead 24 connected to the bump 40a are connected to the ground potential of the relay substrate 26.

In the present embodiment, the electrode sheet 16 laminated on the upper side of the acoustic matching layer 14a wraps around side surfaces of the vibration element array 12a and the acoustic matching layer 14a at the end portion in the longitudinal direction, and is in contact with the bump 40a located on the exposed portion 72. Accordingly, the electrode sheet 16 is connected to the ground potential. Although there is one bump 40a (lead 24) that is in electrical contact with the electrode sheet 16 in FIG. 5, a plurality of bumps 40a may exist in the exposed portion 72, and the plurality of bumps 40a may be in contact with the electrode sheet 16. As shown in FIG. 5, the protective layer 18 extends in the longitudinal direction so as to cover the end portion of the backing 20 in the longitudinal direction. An adhesive is injected into a gap between the electrode sheet 16 in contact with the exposed portion 72 and the protective layer 18.

The outline of the configuration of the ultrasonic probe according to the present embodiment is as described above. In the present embodiment, the lower end portions of the plurality of leads 24 are grouped so as to correspond to the ICs 32, so that the lower end position of each lead 24 (position of the bump 42) and the position of the ball-shaped terminal 44 of the IC 32 are close to each other. Accordingly, the length of the wiring pattern of the relay substrate 26 in the longitudinal direction can be shortened. That is, the wiring pattern of the relay substrate 26 is simplified. Ideally, if the pitch of each lead 24 (bump 42) in the dense group 46 and the pitch of each ball-shaped terminal 44 are the same, the wiring pattern does not need to be drawn in the longitudinal direction in the relay substrate 26.

In general, in the convex two-dimensional ultrasonic probe, since the length in the short direction is considerably shorter than the length in the longitudinal direction, the wiring pattern of the relay substrate 26 is likely to be long, particularly in the longitudinal direction, that is, the wiring pattern of the relay substrate 26 is likely to be complicated. On the other hand, by grouping the leads 24, a degree of difficulty in manufacturing the backing 20 is increased. Therefore, in the present embodiment, by grouping the leads 24 only in the longitudinal direction, the length of the wiring pattern of the relay substrate 26 in the longitudinal direction that is highly demanded for simplification particularly is shortened, and the grouping is not performed in the short direction, thereby preventing an increase in manufacturing difficulty of the backing 20. Particularly, by setting the pitch of each lead in the short direction to be constant, a manufacturing method of a sheet laminated type can be adopted at the time of manufacturing the backing 20. Specifically, the backing 20 can be formed by laminating the sheet-shaped backing base portion 22 in which the lead row 24b having the wiring pattern as shown in FIG. 2 is embedded in the short direction.

Although the embodiment according to the invention has been described above, the invention is not limited to the above embodiment, and various changes may be made without departing from the spirit of the invention.

### REFERENCE SIGN LIST

10 vibrator unit, 12 vibration element, 12a vibration element array, 14 acoustic matching element, 14a acoustic matching layer, 16 electrode sheet, 18 protective layer, 20 backing, 22 backing base portion, 24 lead, 24a lead array, 26 relay substrate, 28 connector, 30 flexible cable, 32 IC, 40, 42 bump, 44 ball-shaped terminal, 46, 46a, 46b, 46c dense group, 48, 48a, 48b inter-group gap, 50, 50a, 50b inter-device gap, 52 upper end section, 54 intermediate section, 56 lower end section, 58 upper transition section, 60 lower transition section, 72 exposed portion.

1. A convex ultrasonic probe, comprising:
a vibration element array that includes a plurality of vibration elements arranged two-dimensionally in a curved direction corresponding to a longitudinal direction and a short direction perpendicular to the longitudinal direction;
a backing that is provided on a lower side of the vibration element array and includes a lead array including a plurality of leads electrically connected to the plurality of vibration elements, wherein an upper side surface of the backing is a curved surface defined by the curved direction and the short direction;
a plurality of electronic devices that are provided on a lower side of the backing; and
a relay substrate that is a substrate extending in the longitudinal direction and the short direction and electrically connects the lead array and the plurality of electronic devices, wherein
lower end portions of the plurality of leads are grouped into a plurality of dense groups according to an arrangement of the plurality of electronic devices at least in the longitudinal direction.

2. The convex ultrasonic probe according to claim 1, wherein
an inter-group gap exists between two adjacent dense groups in the longitudinal direction,
an inter-device gap exists between two adjacent electronic devices in the longitudinal direction, and
an arrangement of a plurality of inter-group gaps existing on an upper side of the relay substrate corresponds to an arrangement of a plurality of inter-device gaps on a lower side of the relay substrate.

3. The convex ultrasonic probe according to claim 1, wherein
the lead array includes a plurality of lead rows arranged in the short direction,
each lead row includes a plurality of leads arranged in the longitudinal direction, and
a pitch between the plurality of lead rows in the short direction is constant.

4. The convex ultrasonic probe according to claim 3, wherein
each lead row includes a plurality of sections arranged in an upper-lower direction with different wiring patterns, and
the plurality of dense groups are formed by the wiring pattern in any one of the plurality of sections.

5. The convex ultrasonic probe according to claim 4, wherein
the plurality of sections include:
an upper end section that is an upper end portion of the lead row and has a radial pattern corresponding to the vibration element array;
an intermediate section that is an intermediate portion of the lead row in the upper-lower direction and has a main wiring pattern which is a parallel wiring pattern;
a lower end section that is a lower end portion of the lead row and has a grouping pattern including the plurality of dense groups which is a parallel wiring pattern;
an upper transition section that is a section between the upper end section and the intermediate section and has an upper transition pattern that connects the radial pattern and the main wiring pattern; and
a lower transition section that is a section between the intermediate section and the lower end section and has a lower transition pattern that connects the main wiring pattern and the grouping pattern.

6. The convex ultrasonic probe according to claim 5, wherein
the radial pattern includes a plurality of lead upper end portions perpendicular to the curved surface.

7. The convex ultrasonic probe according to claim 5, wherein
the grouping pattern includes a plurality of lead lower end portions perpendicular to a horizontal plane defined by the longitudinal direction and the short direction.

8. The convex ultrasonic probe according to claim 1, wherein
the plurality of lead lower end portions are grouped into the plurality of dense groups according to an arrangement of the plurality of electronic devices also in the short direction.

9. The convex ultrasonic probe according to claim 1, wherein
the vibration element array is formed on an intermediate portion excluding both ends on the upper side surface of the backing, and
an electrode sheet that is laminated on an upper side of the vibration element array and is electrically connected to the leads at both ends of the upper side surface of the backing is provided.
